(19) United States
(12) Patent Application Publication
(10) Pub. No.: US 2005/0288644 A1
(43) Pub. Date: Dec. 29, 2005

(54) Wrapping body

(75) Inventors: Satoshi Mizutani, Kagawa (JP); Wataru Yoshimasa, Kagawa (JP); Yuki Noda, Kagawa (JP)

Correspondence Address: DARBY & DARBY P.C., P. O. BOX 5257, NEW YORK, NY, 10150-5257, US

(73) Assignee: Uni-Charm Corporation, Shikokuchuo-shi (JP)

(21) Appl. No.: 11/142,009

(22) Filed: May 31, 2005

(30) Foreign Application Priority Data
May 28, 2004 (JP) 2004-160161

(51) Int. Cl. A61F 13/15

(52) U.S. Cl. 604/385.02

(57) ABSTRACT

There will be provided a wrapping body in which an inter-labial pad and a combinational sheet used together with the inter-labial pad are enclosed in a common external container, allowing a wearer to instantaneously understand the state of using the inter-labial pad and the combinational sheet together. There will be also provided a wrapping body allowing a wearer to recognize the order of using the inter-labial pad and the combinational sheet from the enclosing positions thereof. The wrapping body comprises a first individual wrapping body enclosing the inter-labial pad, a second individual wrapping body enclosing a combinational sheet used together with the inter-labial pad and an external container integrally containing them, in which an indication means indicating the state of combined use of the inter-labial pad and the combinational sheet is provided on the outer surface of the external container.

### CROSS-REFERENCE TO RELATED APPLICATION

This application is based upon and claims the benefit of priority from Japanese Patent application No. 2004-160161 filed on May 28, 2004, the entire contents of which is incorporated herein by reference.

### FIELD OF THE INVENTION

The present invention relates to a common package in which an inter-labial pad and a combinational sheet thereof are combined and, more specifically, it relates to a wrapping container devised such that even a wearer ignorant of an inter-labial pad can understand how to use it at a glance.

### RELATED ART

An inter-labial pad to be inserted to female labia or absorbing menstrual blood has been known generally so far. Since the inter-labial pad is in close contact with a body, and prevents the menstrual blood from diffusion and contact widely to a body, it is sanitary and clean. Further, compared with a tampon to be inserted into a vagina, it also has a feature of causing less psychological resistance upon attachment.

For example, the patent document 1 (Japanese Patent No. 3300372) discloses a wrapping form of an absorptive inter-labial device as the inter-labial pad described above. In the patent document, the absorptive inter-labial device and the sanitary napkin are formed such that they can be inserted on together, and a female sanitary kit wrapping them into a common package is disclosed. According to the wrapping form, a wearer can easily recognize that both of them are used together.

However, in the wrapping form described in the patent document, a wearer ignorant of the inter-labial pad can not understand how to use the inter-labial pad and may possibly be confused. Specifically, it may be a risk that the wearer can not recognize where to insert the inter-labial pad or which of the inter-labial pad or the sanitary napkin is to be inserted on at first.

### SUMMARY OF THE INVENTION

The present invention has been achieved in view of the foregoing problems and it intends to provide a wrapping body capable of instantaneously understanding the method of using an inter-labial pad and a combinational sheet in a state of wrapping the inter-labial pad and the combinational sheet that can be used together with the inter-labial pad in separate individual wrapping containers respectively as individual packaging bodies and incorporating them in an external container as a common package.

More specifically, the invention provides the followings.

(1) A wrapping body incorporating an inter-labial pad as a sanitary article and a combinational sheet used together with the inter-labial pad by wrapping them integrally into an external container, wherein a first individual wrapping body comprising the inter-labial pad incorporated in an individual wrapping container for the inter-labial pad and a second individual wrapping body comprising the combinational sheet incorporated in an individual wrapping container for the combinational sheet are combined each by one or more and incorporated in the external container, and wherein an indication means indicating the state of combined use of the inter-labial pad and the combinational sheet are disposed to the external container at a position that can be recognized by a wearer.

According to the present invention, the inter-labial pad and the combinational sheet used together with the inter-labial pad are integrally wrapped in the external container. Then, the indication means capable of instantaneously recognizing the state of use in which the inter-labial pad and the combinational sheet are used together is provided on the surface of the external container.

The inter-labial pad is inserted between labia and the combinational sheet is attached to shorts. In a case where a wearer does not recognize this, they are tended to be used erroneously, for example, such that the inter-labial pad is placed on the combinational sheet without being inserted between the labia.

According to the present invention, since it can be seen at a glance how to use the inter-labial pad and the combinational sheet together, the erroneous use as described above can be avoided. Those specifically describing the way of use such as instructions for use may be incorporated in the external container, according to the present invention, a user can recognize the state of use in which the inter-labial pad and the combinational sheet even in a case where articles according to the present invention are in a displayed state and the seal can not be opened.

It may suffice that the “indication means” in the invention can recognize the state of using the inter-labial pad and the combinational sheet and includes, for example, an illustration, a photograph, letters or Braille. They may be used alone or in combination.

In the “indication means”, in a case where indication is made only by way of an illustration or photograph, it is preferred that the inter-labial pad is indicated being spaced apart above the combinational sheet. This can instantaneously teach a wearer that two different forms of sanitary articles are incorporated and that they are put on separately.

In a case of indication by an illustration or the like, when it is drawn such that the inter-labial pad is in contact with a body, it can also teach a wearer that the inter-labial pad is situated above the combinational sheet and insert to a body.

(2) The wrapping body according to (1), wherein the indication means related with the incorporated sanitary article is provided to the individual wrapping container for the inter-labial pad and/or the individual wrapping container for the combinational sheet.

According to the present invention, the method of using the inter-labial pad and the characteristics of the inter-labial pad are indicated to the first individual wrapping body for incorporating the inter-labial pad, and the method of using the combinational sheet and the characteristics of the combinational sheet are indicated to the second individual wrapping body for incorporating the combinational sheet, respectively. Accordingly, also after taking out the wrapping body from the external container, the method of using the inter-labial pad, and the combinational sheet etc. can be recognized merely from the first individual wrapping body or the second individual wrapping body.

“Characteristics” may be indicated by an illustration that expresses the function inherent to the inter-labial pad, specifically, an illustration expressing that the inter-labial pad does not displace even how a wearer moves since the inter-labial pad is put to a body. This includes such an illustration as indicating that the inter-labial pad is fixed to a body and a combinational sheet put to the shorts is caused to slide.

Further, numbers describing the order of putting on the inter-labial pad and the combinational sheet may be added. With such a constitution, even a wearer who does not understand that the inter-labial pad inserted between the labia is detached from the inter-labial space when she opens legs excessively, can avoid the situation that the inter-labial pad is detached from the inter-labial space during attachment of the combinational sheet as a result of putting on the combinational sheet after putting the inter-labial pad to the body.

(3) The wrapping body according to (1) or (2), wherein the wrapping body further comprises a first intermediate wrapping container incorporating only the first individual wrapping bodies in plurality and a second intermediate wrapping container incorporating only the second individual wrapping bodies in plurality, and the indication means showing the method of use provided to the incorporated sanitary article is disposed to the first intermediate wrapping container and/or the second intermediate wrapping container.

According to the present invention, the inter-labial pad and the combinational sheet are wrapped by three types of wrapping containers, that is, individual wrapping containers each incorporating the inter-labial pad and the combinational sheet, intermediate wrapping containers for incorporating the individual wrapping bodies in plurality in a state of incorporating the inter-labial pads and the combinational sheets in the individual wrapping containers respectively, and an external container incorporating the respective intermediate wrapping bodies in a state incorporating the individual wrapping bodies by one or more respectively. Then, an indication means indicating the method of using the inter-labial pad and the combinational sheet to be incorporated is provided to the intermediate wrapping container. Accordingly, the indication means can be applied in a range wider than that of the individual wrapping container, and the characteristics, etc. can be recognized merely by observing the intermediate wrapping container without observing the explanation applied respectively to the individual wrapping containers.

(4) A wrapping body in which a first individual wrapping body incorporating an inter-labial pad and a second individual wrapping body incorporating a combinational sheet used together with the inter-labial pad are incorporated in combination each by one or more in an external container, wherein a transparent region is formed to the external container through which at least a portion of the incorporated first individual wrapping body and second individual wrapping body can be visually recognize (see-through).

According to the present invention, since a portion of the external container is made transparent, the inside can be seen. Therefore, the contents can be confirmed without opening the seal and even a beginner purchase the article while feeling at ease.

Further, in the invention, an illustration or the like showing the state of using the inter-labial pad and the combinational sheet can be provided in addition to the transparent region. Thus, the wearer can recognize, before purchase, to which of the incorporated sanitary articles the remark for the product drawn on the outer surface corresponds without opening the seal of the external container.

(5) A wrapping body according to (4), wherein the transparent region has a design schematically expressing the convex and concave profile of a body portion to which the inter-labial pad is put.

According to this embodiment of the present invention, a body portion to which the inter-labial pad is put is traced in the transparent region capable of seeing-through the content. Therefore, a wearer can image the state of putting on without opening the seal. Further, by expressing the body shape in a simplified form, a pattern and the function described above can be provided together.

(6) A wrapping body according to (5), wherein the design is constituted by providing a not transparent or semi-transparent portion in the transparent region.

According to this embodiment of the present invention, convex and concave profile of the body portion is schematically expressed by making a portion of the transparent portion not transparent or semi-transparent. Therefore, the portion can be made more illustrative.

(7) A wrapping body according to (5) or (6), wherein the first individual wrapping body and the second individual wrapping body are each in a substantially plate shape, the convex and concave profile of the body portion traces labia, the first individual wrapping body is arranged so as to be up-righted relative to a convex portion corresponding to a pudendal slit, and the second individual wrapping body is arranged so as to be turned sideway relative to the convex portion.

According to this embodiment of the invention, the state of use (state of wearing) can be recognized in a state as packaged. That is, while the inter-labial pad is used being put between the labia, the first individual wrapping body incorporating the inter-labial pad is contained in the external container in the convex/concave portion tracing the labia such that as if it were put to the concave portion. Further, since the combinational sheet is put to shorts, it is positioned vertical to the pudendal slit. According to the invention, since the combinational sheet is incorporated in a state it is turned down, this can associate the state of wearing. That is, since the shape of the see-through region is in a shape of tracing the convex/concave profile of the body, a wearer can understand, before purchase, which part of the body and how the inter-labial pad is put on.

Since the first individual wrapping body is disposed at the upper stage and the second individual wrapping body is disposed at the lower stage in the external container, both of them can be seen visually in a state nearer to the state of wearing.

(8) A wrapping body in which a first individual wrapping body for incorporating an inter-labial pad to be used being inserted between labia and a second individual wrapping body for incorporating a combinational sheet to be used together with the inter-labial pad are incorporated in combination each by one or more in an external container, wherein the positions for incorporating the first individual wrapping body and the second individual wrapping body are determined in relation with the order of using the inter-labial pad and the combinational sheet.

According to this embodiment of the invention, a wearer can access the inter-labial pad and the combinational sheet in accordance with the sequence of their use. That is, since the wearer is guided to put the inter-labial pad to a body after putting of the combinational sheet regarding the procedure for use, even a wearer who is ignorant of or not familiar with the article, can follow an exact putting procedure with no confusion.

(9) A wrapping body according to (8), wherein the combinational sheet is put on prior to the inter-labial pad, the external container has a seal-opening means for opening the seal of the external container and the second individual packaging is incorporated being disposed at a position nearer to the seal-opening means than the first individual wrapping body.

According to the invention, the second individual packaging body incorporating the combinational sheet is disposed near the seal-opening means that forms a seal-opening port of the external container. Therefore, the wearer can spontaneously follow the exact putting order such as at first accessing the second individual packaging body after opening the seal of the external container, taking out the combinational sheet and then accessing the first individual packaging body incorporating the inter-labial pad. Accordingly, in a case of putting the inter-labial pad and the combinational sheet, she can avoid the risk of putting the combinational sheet after previous putting the inter-labial pad to the body thereby detaching the inter-labial pad from the body during putting of the combinational sheet.

As described above, according to the invention, even for a packaging body incorporating the inter-labial pad and the combinational sheet used together in one external container, since the indication means showing the relation between both of them is applied, even a wearer ignorant of the inner-labial pad can instantaneously understand the method of use, and the risk of erroneous use can be decreased.

Further, a reference that associates the previous attachment of the combinational sheet before the inter-labial pad is drawn to the individual packaging bodies incorporating the inter-labial pad and the combinational sheet and the intermediate packaging container for covering the individual packaging bodies each in plurality. This enables the wearer to use the article correctly with reference to the drawn remark after taking out containers for covering one or plural inter-labial pads or combinational sheets out of the external container as a common packaging even when she forgets the putting order of the inter-labial pad and the combinational sheet while she carries about them.

Further, by relating the incorporation of them with the order of putting inter-labial pad and the combinational sheet, the wearer can be guided spontaneously to follow the exact putting procedure of putting the combinational sheet and then putting the inter-labial pad to the body.

### BRIEF DESCRIPTION OF THE DRAWINGS

FIG. 1 is a perspective view of a packaging body according to a first embodiment of the invention,

FIG. 2 is a view of a packaging body according to a second embodiment of the invention,

FIG. 3 is a view of a packaging body according to a third embodiment of the invention,

FIG. 4 is a perspective view of a packaging body according to a forth embodiment of the invention,

FIG. 5 is a view showing another example of a packaging body according to a fifth embodiment of the invention,

FIG. 6A is a perspective view of a first individual packaging body incorporated in a packaging body according to the embodiment of the invention,

FIG. 6B is a view in a case where the seal of the first individual packaging body is opened,

FIG. 7A is a perspective view of a second individual packaging body incorporated in a packaging body according to the first embodiment,

FIG. 7B is a view in a state of opening the seal of the second individual packaging body,

FIG. 8 is a perspective view for a first intermediate body incorporated in a packaging body according to an eighth embodiment of the invention,

FIG. 9 is a perspective view for a modified example of the second individual packaging body,

FIG. 10 is a perspective view for a modified example of the first individual packaging body,

FIG. 11 is a perspective view showing a modified example of the first intermediate packaging body,

FIG. 12 is a perspective view for a packaging body according to a sixth embodiment of the invention,

FIG. 13 is a view according to a seventh embodiment of the invention,

FIG. 14 is a view according to an eighth embodiment of the invention, and

FIG. 15 is a view according to a ninth embodiment of the invention.

### DESCRIPTION OF THE SYMBOLS

1: inter-labial pad

2: combinational sheet

3A˜3I: external container

4: labia

5: finger

6: transparent see-through region

7: corrugated not transparent region

8: first intermediate packaging container

9: perforations

10: illustration portion

11, 11A, 11B: first individual packaging body

12, 12A, 12B: second individual packaging body

18: first intermediate packaging body

19: second intermediate packaging body

21: first individual packaging container

22: second individual packaging container

23,24: tape

200A-200I: packaging body

### DESCRIPTION OF THE PREFERRED EMBODIMENT

Each of the preferred embodiments of the invention is to be described with reference to the drawings. In the following explanation for the preferred embodiments, identical constituent factors carry same reference numerals for which descriptions are to be omitted or simplified.

[Packaging Form]

In the invention, individual packaging bodies incorporating the inter-labial pad and the combinational sheet respectively are combined at the outside thereof. That is, they constitute a form other than that the inter-labial pad and the combinational sheet are incorporated in one individual packaging body. Specific examples include a case where one individual packaging body for an inter-labial pad and one individual packaging body for a combinational sheet are incorporated in an external container, a case where one individual packaging body for an inter-labial pad and one individual packaging body for a combinational sheet are incorporated being joined by means of adhesives, etc. in an external container, a case where plural individual packaging bodies for inter-labial pads and plural individual packaging bodies for combinational sheets are incorporated each by the equal number or different number in an external container, and a case where plural individual packaging bodies for inter-labial pads or plural individual packaging bodies for combinational sheets are incorporated in an intermediate packaging container and further incorporated in an external container. As described above, since the individual packaging bodies are combined at the outside thereof respectively, even in a case where putting of the inter-labial pad is failed to leave an excessive combinational sheet, it is not necessary to open the seal of the combinational sheet and it can be stored in a sanitary manner.

By making the number equal between the inter-labial pads and the combinational sheets sealed in the external container with each other, the beginner can recognize again that the inter-labial pad is not used alone but should be used together with the combinational sheet. Further, in a case where the number of the inter-labial pads is made larger than that of the combinational sheets, even when a wearer who is ignorant of or not familiar with the use should fail putting of the inter-labial pad making it wasteful, since there are spare inter-labial pads, it can avoid an undesired situation of resulting in a number of combinational sheets which are left used.

[Packaging Body]

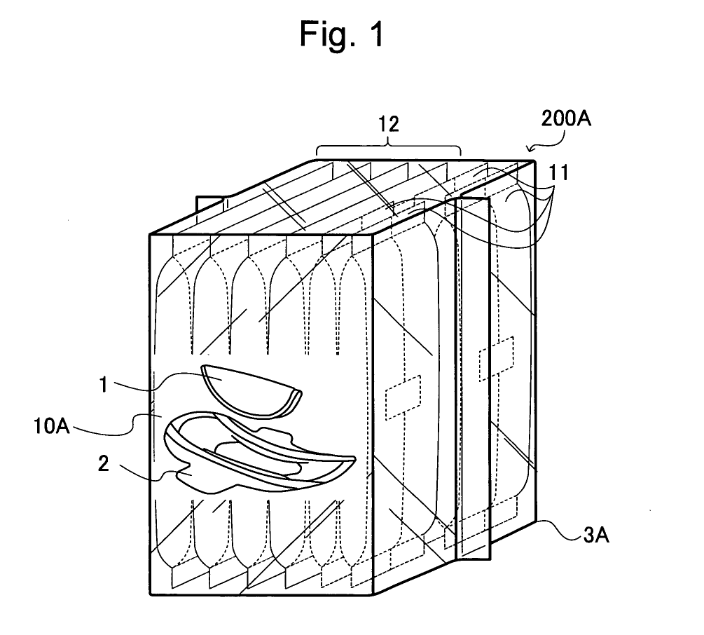

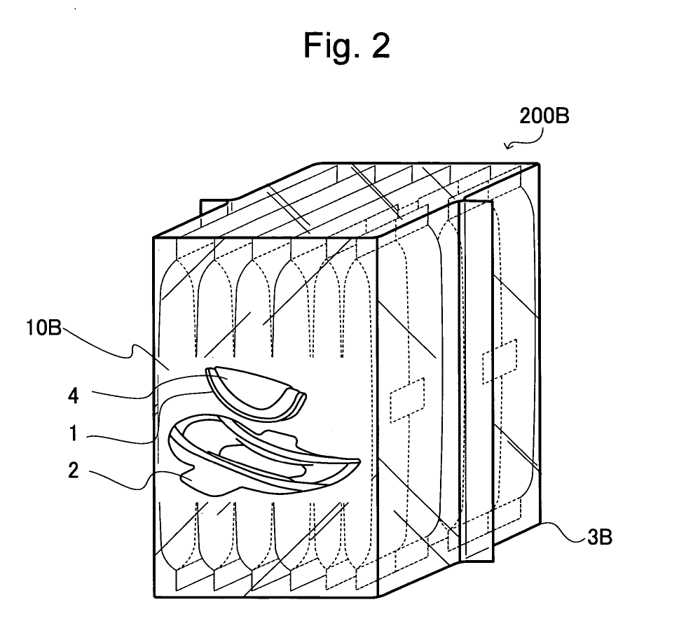

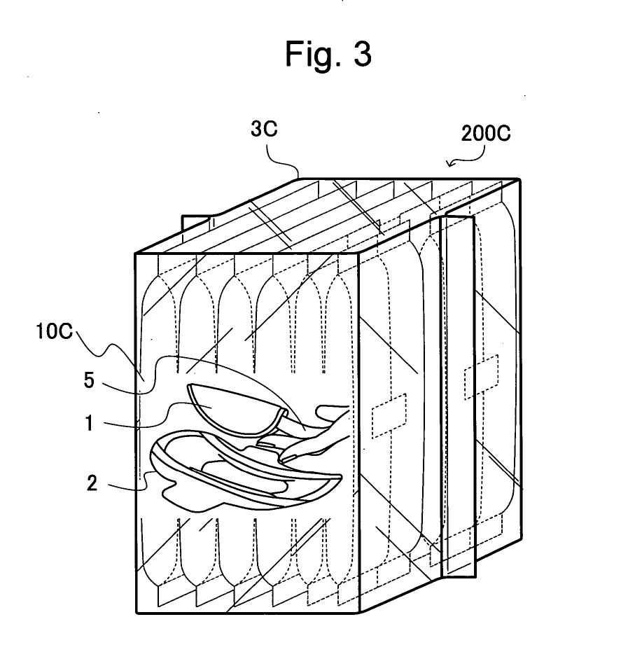

At first, the first and the second embodiments of the packaging body according to the invention are to be described. FIG. 1 is a view showing a packaging body 200A according to a first embodiment of the invention. The packaging body 200A has an external container applied with an illustration showing the state where inter-labial pads and combinational sheets are spaced apart. FIG. 2 is a view showing a packaging body 200B according to a second embodiment of the invention. The packaging body 200B has an external container applied with an illustration expressing the state of putting an inter-labial pad to a body. FIG. 3 is a view showing a packaging body 200C according to a third embodiment of the invention. The packaging body 200C has an external container applied with an illustration showing the state of putting the inter-labial pad with fingers.

As shown in FIG. 1, a first inter-labial packaging bodies 11 in which inter-labial pads each sealed in an individual packaging container for an inter-labial pad and second individual packaging bodies 12 in which intermediate sheets each sealed in an individual packaging container for a combinational sheet are sealed, each by four, in an up-righted state in an external container 3A. An illustration portion 10A is provided to the surface of the external container 3A, and the inter-labial pad 1 and the combinational sheet 2 are traced in the illustration portion 10A. The inter-labial pad 1 is positioned above the combinational sheet 2 and they are drawn as being spaced apart from each other. Accordingly, this makes it clear that the inter-labial pad 1 is not connected with the combinational sheet but they are separated from each other.

As shown in FIG. 2, an illustration portion 10B that traces an inter-labial pad 1 and a combinational sheet 2 is provided to the outer surface of an external container 3B in which the inter-labial pad 1 is drawn in a state in contact with the labia 4. Therefore, it is apparent that the inter-labial pad is put between the inter-labia.

While the labia are traced in this embodiment, this is not restrictive but an illustration portion in which the entire lower half body of a female is drawn may be provided so long as a concave portion expressing a pudendal slit or labia is drawn. By the provision of the illustration portion in such a state, a wearer can understand that the inter-labial pad is put to the body.

An illustration portion 10C tracing the inter-labial pad 1 and the combinational sheet 2 is provided also to the outer surface of the external container 3C shown in FIG. 3 and, further, an illustration in a state where a finger is inserted in the inter-labial pad 1 is drawn. The drawing of the finger 5 can estimate the size of the inter-labial pad 1 and since the illustration showing the state of inserting the finger 5 in the inter labial pad 1 is drawn, it can also show the way of putting the inter-labial pad 1.

An illustration for shorts may further be drawn to the external container 3 so as to express that the combinational sheet 2 is put to shorts. Further, so that a wearer can recognize that the inter-labial pad is to be put to the body while the combinational sheet is to be put to shorts, both the labia and the shorts may be expressed as an illustration showing the state of putting both of them. Further, an illustration expressing the effect inherent to the inter-labial pad may be provided.

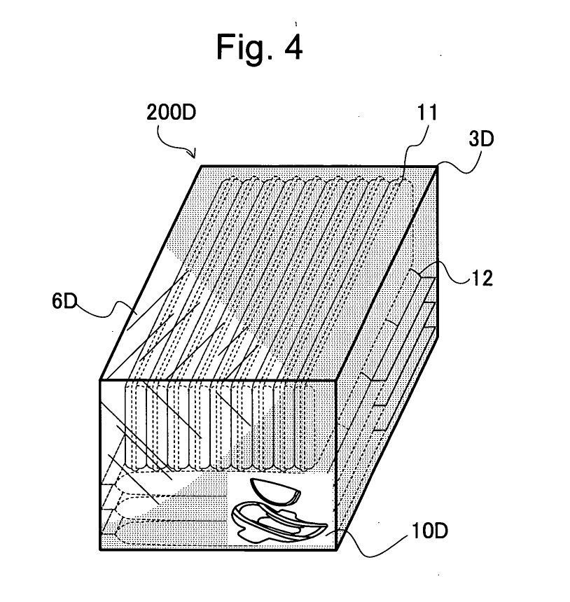

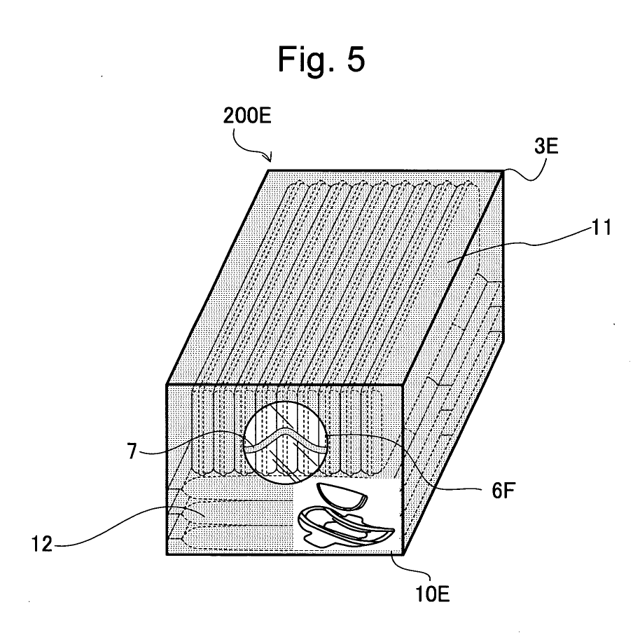

Then, fourth and fifth embodiments of the invention are to be described. FIG. 4 shows a packaging body 200D of the fourth embodiment of the invention. The packaging body 200D comprises an external container having a transparent region through which the contents can be seen. FIG. 5 is a view showing a packaging body 200E according to the fifth embodiment of the invention. The packaging body 200E comprises an external container in which the shape of the body is traced by the provision of a not transparent region in the transparent region.

As shown in FIG. 4, a packaging body 200D is formed in a state where fist individual packaging bodies 11 each incorporating an inter-labial pad are arranged by the number of nine in the upper stage and second individual packaging bodies 12 in which a combinational sheet is shield in the individual packaging container are arranged by the number of three on the lower stage are sealed in an external container 3D. Then, a transparent see-through region 6D is provided to the external container 3D, so that the state of the contents can be observed visually. An illustration portion 10D is provided to a portion other than the transparent region 6D. Accordingly, by observing the illustration portion 10D and further observing the transparent region 6D, the state of use can be recognized more exactly.

As shown in FIG. 5, a circular transparent region 6E that can not be see-through is provided to the external container 3E, and a corrugated not transparent region 7 is disposed in the transparent region 6E. The not transparent region 7 shows a convex/concave profile of the body and shows a pudendal slit or labia in this embodiment, and it forms a convex shape of laterally overriding the center line for the substantially circular transparent region 6E. Then, through the transparent region 6E, the state where the first individual packaging body 11 is up-righted along the inside of the concave shape and the second individual packaging body 12 is turned down can be seen. Accordingly, the state of the inter-labial pad and the combinational sheet put to the body can be recognized without opening the seal of the external container 3.

[Individual Packaging Body and Intermediate Packaging Body]

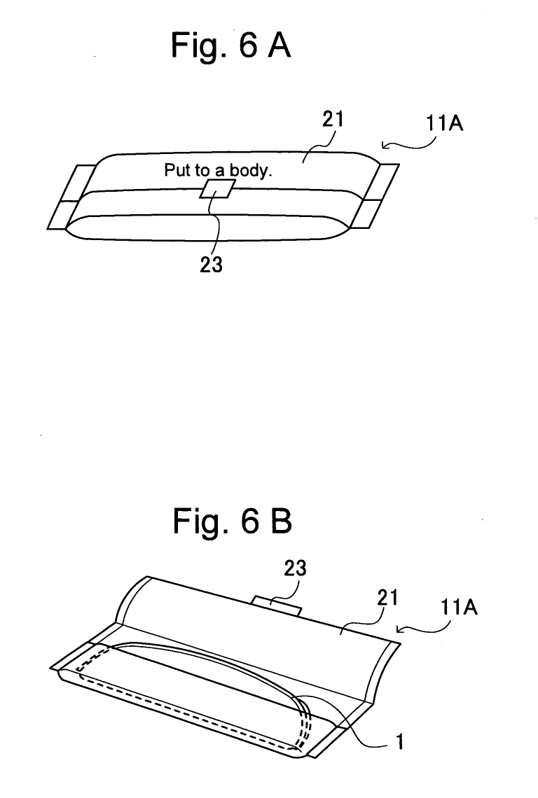

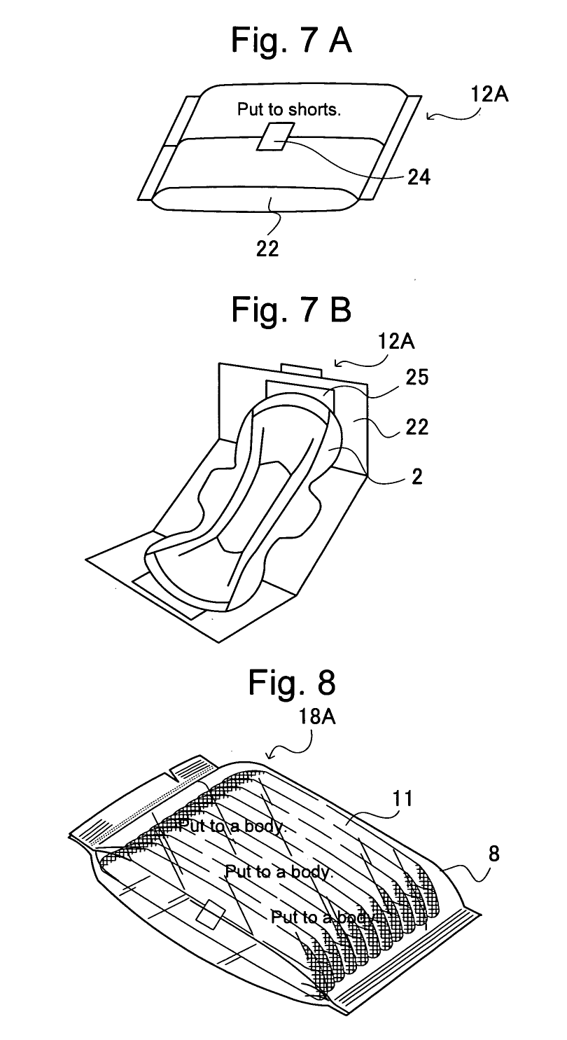

Then the individual packaging body and the intermediate packaging body are to be described. FIG. 6A is a view showing a first individual packaging body and FIG. 6B is a view when the seal of the first individual packaging body is opened. FIG. 7A is a view showing a second individual packaging body and FIG. 7B is a view when the seal of the second individual packaging body is opened. FIG. 8 shows an intermediate packaging body according to an eighth embodiment of the invention to be described later.

FIG. 6A shows a first individual packaging body 11A which comprises an inter labial pad 1 and a first individual packaging container 21 for incorporating the pad as shown in FIG. 6B. A remark is put on by means of words “put to a body” on the outer surface of the first individual packaging container 21. The remark is not restricted to the words described above so long this associates that the inter-labial pad is put to the body. An illustration as well as other words may also be drawn. Further, the position for the remark is not restricted to the outer surface of the first individual packaging container 21 but it may be at the inner surface of the first individual packaging container 21, at the tape 23, or on the side of the inter labial pad 1 facing the garment, etc.

FIG. 7 is a view showing a second individual packaging body 12A incorporating a combinational sheet and, as shown in FIG. 7B, the second individual packaging body 12A comprises a combinational sheet 2 and a second individual packaging container 22 incorporating the sheet. A remark consisting of words “put to shorts” is applied to the outer surface of the second individual packaging container 22. The remark is not restrictive so long as this associates that the combinational sheet is put to shorts, and an illustration as well as other words may also be drawn. Further, the position for the remark is not restricted to the outer surface of the second individual packaging container 22 but it may be on the inner surface of the second individual packaging container 22, at the tape 24, release paper 25 and on the surface of the combinational sheet 2 facing the garment.

Since the remark is applied on the first individual packaging body or the second individual packaging body per se as in FIG. 6 or FIG. 7 described above, even in a case where a wearer forgets the order of putting on of the inter-labial pad and the combinational sheet after taking them out of the external container or the intermediate container covering a plurality of them while she caries about the articles, she can correctly use them with reference to the remark.

In a case where a wearer stores the combinational sheet and other sanitary napkins together, it may be a worry that she can not instantaneously distinguish the inter-labial pad and the combinational sheet to be used in combination with the pad and becomes confused. Then, it is preferred that a common remark is applied to the individual packaging body for the inter-labial pad and the individual packing body for the combinational sheet. Specifically, this include, for example, that the color for the individual packaging bodies for the inter-labial pad is identical with that of the combinational sheet or the tape, that a common pattern such as a rhombic shape, a polka-dotted shape or item number is drawn in common, and that they are made of a common material.

Further, as shown in FIG. 8, in a case where plural first individual packaging bodies 11 which are incorporated by air tight sealing in the first intermediate packaging container 8 made of a transparent air tight material are used as a first intermediate packaging body 18A, a remark consisting of words of “put to a body” may also be attached to the outer surface of the first intermediate packaging container 8. The remark is used with no restriction so long as this associates that the inter-labial pad is put on the body and other words or illustrations may also be drawn. Further, since the inter-labial pad can be tightly closed in a state of filling air by the first intermediate packaging container 8, even when the external pressure exerts, the impact on the inter-labial pad can be moderated by air. As shown FIG. 14 to be described subsequently, the first intermediate packaging body can be incorporated in the external container.

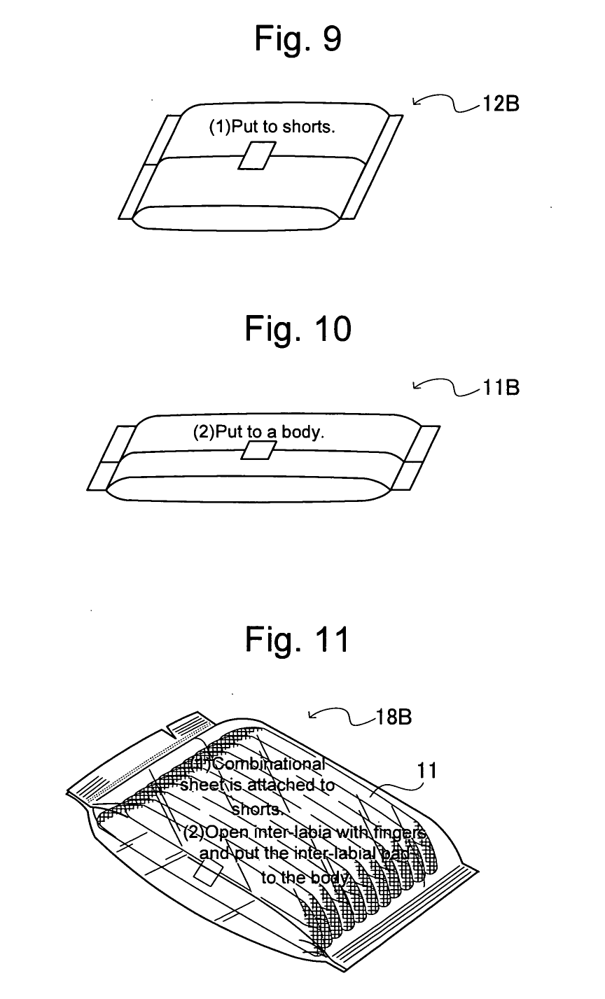

Then, modified examples for the individual packaging body and the intermediate packaging body are to be described. FIG. 9 is a modified example of a second individual packaging body, FIG. 10 is a perspective view of a modified example of a first individual packaging body, and FIG. 11 is a view showing a modified example of an intermediate packaging body incorporating the first individual packaging body.

As shown in FIG. 9, a remark consisting of words of “(1) put to shorts” numbered as (1) showing the putting order is described on the second individual packaging body 12B. Accordingly, it is possible to recognize that the combinational sheet is at first put. The remark is used with no limitation so long as this associates that the combinational sheet is put on before the inter-labial pad and other words or illustrations may also be drawn. The position for the remark is not restricted only to the outer surface of the individual packaging body but it may be on the inner surface of the individual packaging body, on the tape, on the releasing paper and on the surface of the combinational sheet on the side of a garment.

In FIG. 10, a remark consisting of words “(2) put to the body” is described on the outer surface of the first individual packaging body 11B. This can indicate that the inter-labial pad is put on after putting on the combinational sheet. The position for the remark is not restricted only to the outer surface of the individual packaging body but it may be also on the inner surface of the individual packaging, on the tape, on the releasing paper and on the surface of the inter-labial pad on the side of a garment.

Then, FIG. 11 shows a first intermediate packaging body 18B in which plural first individual packaging bodies 11 are sealed. A remark consisting of words “(1) combinational sheet is attached to shorts”, “(2) open inter-labia with fingers and put the inter-labial pad to the body” are described to the outer surface of the first inter-mediate body 18B. As described above, both the numbering showing the putting order and words showing the putting procedure or putting method can be described.

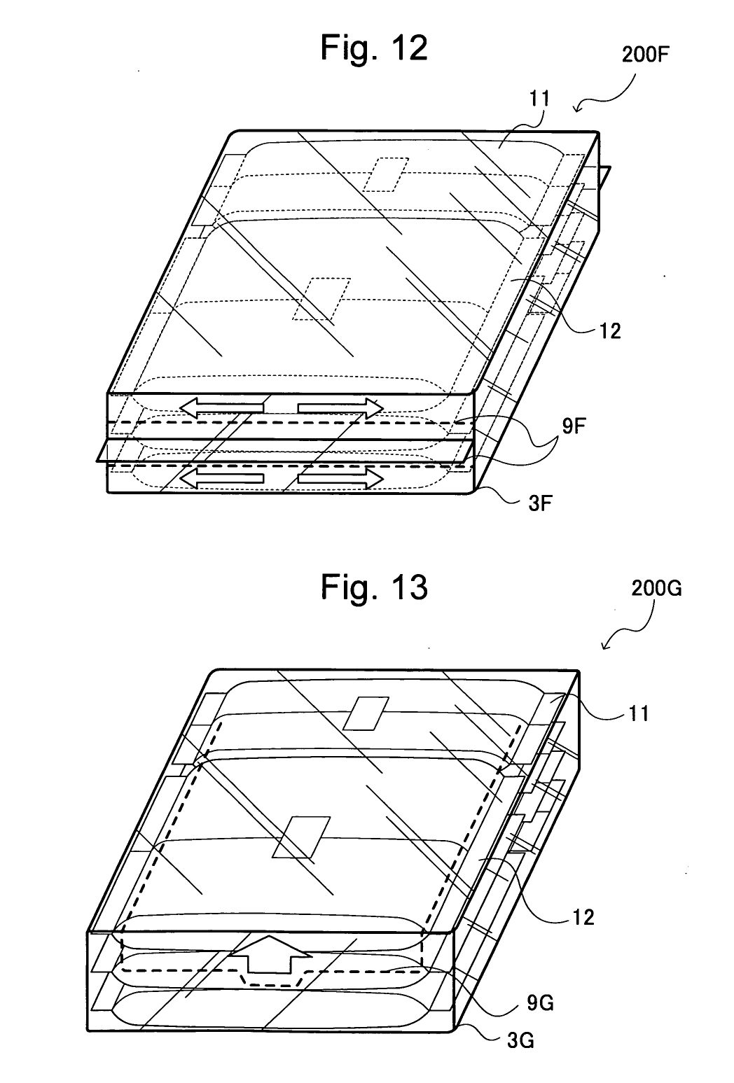

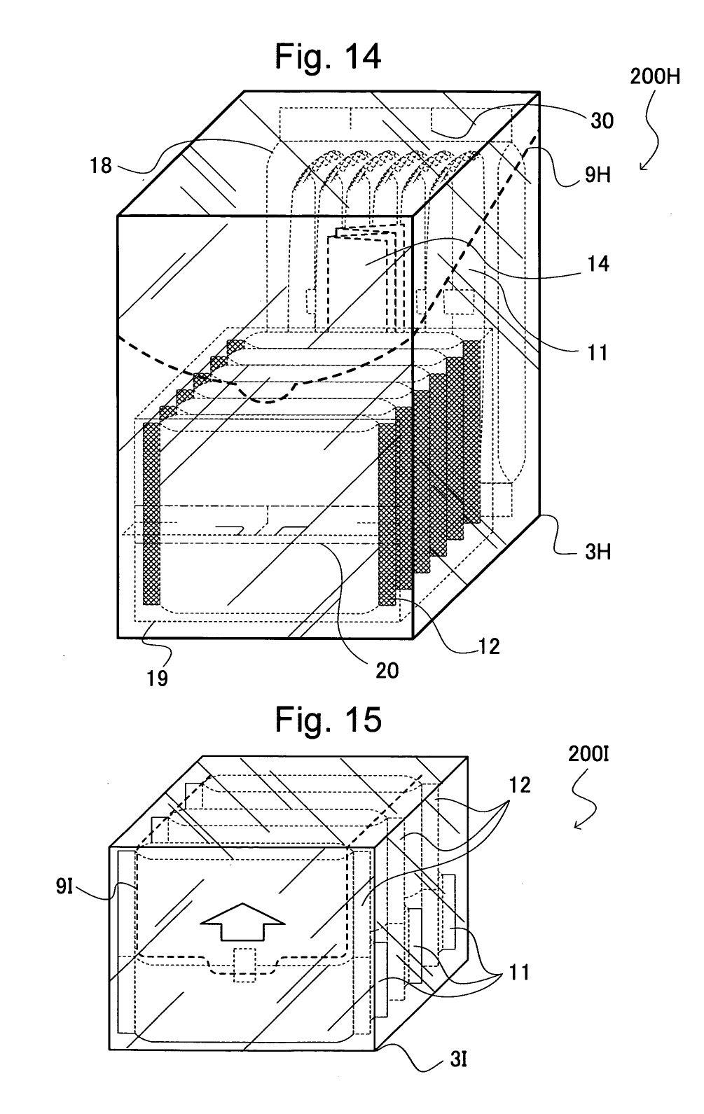

Then, sixth, seventh, eighth and ninth embodiments according to the invention are to be described. FIG. 12 is a view showing a packaging body 200F according to the sixth embodiment of the invention. The packaging body 200F is a packaging body having perforations that form a seal-opening means for the external container. FIG. 13 is a perspective view showing a packaging body 200G according to the sixth embodiment of the invention. The packaging body 200G is a packaging body having an external container provided with perforations from the front surface to the top surface of the external container. FIG. 14 is a view showing a packaging body 200H according to the eighth embodiment of the invention. The packaging body 200H has an external container in which perforations are formed from the frontal surface to the lateral surface of an external container. FIG. 15 is a view showing a packaging body 200I according to the ninth embodiment of the invention. The packaging body 200I is a packaging body in which a first individual packaging bodies and second individual packaging bodies are incorporated alternately.

FIG. 12 is a perspective view showing the packaging body 200F according to the sixth embodiment of the invention. The packaging body 200F is a packaging body in which perforations 9F are formed from the frontal surface to the lateral surface of an external container 3F in which first individual packaging bodies 11 are sealed by the number of three on the back surface, second individual packaging bodies 12 are sealed by the number of three in the frontal surface, each in a laid-down the state, in an external container 3F. Then, the seal-opening means for the external container 3F is constituted with perforations 9F, and the second individual packaging body 12 is situated in the vicinity thereof. Therefore, a laterally long take-out port is formed by seal-opening the perforations 9F outwardly on both leftward and rightward directions, and the individual packaging body 12 in the turned down state can be taken out of the laterally long take out port. Accordingly, an operation of putting a combinational sheet to shorts and then putting the inter-labial pad between inter-labia can be conducted smoothly. The seal-opening means for the seal-opening port may be formed also by half-cutting in addition to the perforations.

As shown in FIG. 13, perforations 9G may be formed from the frontal surface to the top surface of an external container 3G. Then, a second individual packaging body 12 is provided such that it is on the side of the wearer when the seal is opened along the perforations 9G. Then, the second individual packaging body 12 can be at first taken out and then the first individual packaging body 11 on the depth can also be taken out smoothly.

In FIG. 14, perforations 9H are formed from the frontal surface to the lateral surface of an external container 3H. Since the second intermediate packaging body 19 incorporating a second individual packaging body 12 is provided such that it is on the side of the wearer when the seal is opened, the wearer can at first take out the second intermediate packaging body 19. In the intermediate packaging body 19, the second individual packaging body 12 can be taken out by opening the seal of the perforations 20 as the seal-opening means thereby forming an seal-opening port. In the first intermediate packaging body 18, the first individual packaging body 11 is taken out by opening the seal from the notch 30 as the seal-opening means thereby forming a seal-opening port.

In a case of containing an instruction for use, it is preferably disposed at a position where the existent thereof can be recognized at the same time with the seal-opening of the external container. Specifically, while it may be adhered, for example, by means of adhesives to the outside of the external container or at the back of the top surface of the external container, in a case where it is bonded, for example, by means of adhesives, the external container may possibly be fractured upon peeling. Accordingly, as shown in FIG. 14, in a preferred example, an instruction 14 for use is put between the first intermediate packaging body 18 and the second intermediate packaging body 19 while making the height of the second individual packaging body 12 smaller than that of the first individual packaging body 11 that incorporates the inter-labial pad.

In FIG. 15, first individual packaging bodies 11 and second individual packaging bodies 12 are alternately arranged from the seal-opening means formed by perforations 91 sequentially as: a second individual packaging body 12, a first individual packaging body 11, a second individual packaging body 12, a first individual packaging body 11 and sealed in an external container 31. This can suggest more reliably the order of using the combinational sheet and the inter-labial pad. In this case, the first individual packaging body 11 and the second individual packaging body 12 may be joined each by one, for example, with adhesives so as to form a one set.

<Inter-Labial Pad>

The inter-labial pad is attached being put between the wearer's labia and, since the labia are an extremely delicate portion, the pad is preferably longitudinal and flexible.

The shape of the inter-labial pad is not particularly restricted so long as it is a shape conforming the female labia such as an elliptic shape, a hourglass shape or a droplet shape. The total size for the outer profile is, preferably, from 50 to 180 mm and, more preferably, from 80 to 120 mm in the longitudinal direction. Further, it is preferably from 30 to 100 mm and, preferably, from 50 to 80 mm in the shorter direction.

The constitution of the inter-labial pad includes those comprising a liquid pervious top sheet having a property of allowing a liquid to permeate therethrough as a cover member on the surface in contact with a body in the state of wearing, a less liquid pervious back sheet having a nature of not substantially allowing a liquid to permeate therethrough as a cover member opposite to the surface in contact with the body and an absorbent body interposed between the top sheet and the back sheet. In a case of incorporating the inter-labial pad in the individual packaging container, it is sealed in a state being bent along the longitudinal center line such that the back sheets are opposed to each other and a tape for securing the same is preferably disposed in the seal-opening portion of the individual packaging container.

For the top sheet disposed on the side in contact with the body, those materials which are water pervious and give less stimulation to skins are used. They include those materials using non-woven fabrics obtained by a manufacturing method such as point bonding or through air, etc. alone or in a composite form. Among the materials those mainly comprising at least hydrophilic cellulosic fibers are preferred in view of the affinity with the inner-labial inner walls so that displacement is not caused between the inter-labial pad and the labial inner wall to give a foreign-body sensation to a wearer. Specifically, spun laced non-woven fabrics obtained by mixing from 5 to 30% of natural cotton and from 70 to 95% of rayon or acetate, conditioning to a range from 20 to 50 g/m², then entangling fibers to each other by water jet entanglement followed by drying and conditioning the thickness ranging from 0.3 to 1.0 mm. The thread material used in these case is selected within a range from 1.1 to 6.6 dtex and from a range of 15 to 60 mm for natural cotton and from a range of 25 to 51 mm for rayon or acetate. Further, they may be also films having perforation apertures or fiber layers laminated with films and having perforation apertures.

As the back sheet disposed on the surface opposite to the surface in contact with a body, those capable of preventing menstrual blood kept in the absorbent body from leaking to the outside of the inter-labial pad may be used. Further, by the use of moisture permeable materials, steaming during wearing can be decreased and uncomfortable feeling during wearing can be reduced. The materials for the less water permeable sheet include films constituted with polyethylene, polypropylene, polyethylene terephthalate, polyvinyl alcohol, polylactic acid, or polybutyl succinate, non-woven fabric, paper and laminate materials thereof at a thickness from 15 to 60 μm. Further, the materials may also be air permeable films obtained by filling inorganic fillers in resin and applying stretching. Specifically, they include films mainly comprising low density polyethylene resin and conditioned to a range of a basis weight of from 15 to 30 g/m² and, further, air permeable films controlled within a range of an apertured area ratio of from 10 to 30% and an aperture diameter of from 0.1 to 0.6 mm. Examples of the non-woven fabrics include spun bonded non-woven fabric, point bonded non-woven fabric, and through air non-woven fabric, which may be applied with a water repelling treatment. Among them, SMS non-woven fabrics containing melt blown fibers constituted with ultrafine fibers and with extremely small inter-fiber distance (three layered constitution of: spun bonded fiber layer/melt blown fiber layer/spun bonded fiber layer) are preferred. In this case, it is preferred to constitute within the range of the basis weight of from 5 to 15 g/m²/from 1 to 10 g/m²/from 5 to 15 g/m².

[Absorbent Body]

As the absorbent body for absorbing the body fluid such as menstrual blood, pulp, chemical pulp, rayon, acetate, natural cotton, super absorbent polymer, fibrous polymeric absorbent body, synthetic fibers and other materials alone or as a mixture of them, bulky, less deforming and giving less chemical stimulations are preferred. Specifically, they include non-woven fabric sheets having a basis weight of from 50 to 500 g/m² and a volume of from 1 to 20 mm obtained by embossing-sheeting fibers formed by laminating, at a mixing ratio, of from 95 to 60% of rayon or acetate selected within a range from 1.1 to 6.6 dtex, from 1 to 10% of polymeric absorbent body and from 4 to 30% of natural cotton. For the manufacturing method, they may be sheeted by an air-laid method, spun lacing method, paper making method, etc. and, further, applying needling or embossing by passing between rolls having a dot-like or lattice-like shape. An emboss area ratio is, preferably, within a range of 0.3 to 60% in the case of embossing using dot-like emboss patterns.

<Individual Packaging Body for Inter-Labial Pad>

The materials for the individual packaging container for containing an inter-labial pad and forming an individual packaging body include films constituted with polyethylene, polypropylene, polyethylene terephthalate, polyvinyl alcohol, polylactic acid, or polybutyl succinate, or non-woven fabric, paper and laminate materials thereof at a thickness of from 15 to 60 μm. Specifically, they include films formed by mixing from 0 to 80% of a low density polyethylene resin, and from 100 to 20% of a high density polyethylene, and controlling them to a basis weight within a range from 15 to 35 g/m². Further, films applied with stretching for increasing the resin orientation may also be used. Examples of the non-woven fabrics include spun bonded non-woven fabrics, point bonded non-woven fabrics or through-air non-woven fabrics, etc., which may be applied with a water repelling treatment. Among them, SMS non-woven fabrics containing melt blown fibers constituted with ultrafine fibers and with extremely small inter-fiber distance are preferred. In this case, the basis weight for each of the layers is preferably constituted within a range from 5 to 15 g/m²/1 to 10 g/m²/5 to 15 g/m².

Further, it is preferred that the individual packaging body can shield the color of the menstrual blood absorbed in the inter-labial pad, and a pigment may be mixed within a range from 0.2 to 10%, or ink, etc. may also be printed.

<Combinational Sheet>

The combinational sheet to be used together with the inter-labial pad includes those provided with adhesives for attachment being bonded to shorts, which is set at least larger than the size of the rear face of the inter-labial pad during wearing and capable of absorbing and retaining menstrual blood overflown from the inter-labial pad during wearing.

The shape of the combinational sheet is not particularly limited so long as it is a shape conforming the female body and the shape of the shorts such as, a rectangular shape, an elliptic shape or an hourglass form or shape having attachment of so-called wings for preventing displacement relative to shorts. The total size for the outer profile is, preferably, from 100 to 500 mm and, more preferably, from 170 to 350 mm in the longer direction, while it is, preferably, from 30 to 200 mm and, more preferably, from 40 to 180 mm in the shorter direction.

The constitution of the combinational sheet includes a constitution comprising a liquid pervious top sheet having a properly of allowing a liquid to permeate therethrough as a cover member, a liquid less pervious back sheet having a properly of not substantially allowing a liquid to permeate therethrough as a cover member, an absorbent body interposed between the top sheet and the back sheet and adhesives provided to the rear face of the back sheet for preventing displacement relative to the shorts. Further, in a case of containing in the individual packaging container, the combinational sheet is sealed, for example, in a 3-folded or 4-folded state to the inner surface and a seal-openable portion of the individual packaging container is provided with a tape and secured. Further, for easy releasing of the combinational sheet from the individual packaging body, a releasing treatment with silicon or the like is applied to the inner surface of the individual packaging container, or a sheet applied with a releasing treatment may be disposed between the combinational sheet and the individual packaging body.

[Absorbent Body]

Preferably, the absorbent body comprises pulp, chemical pulp, rayon, acetate, natural cotton, polymeric absorbent body, fibrous polymer absorbent body, synthetic fibers alone or as a mixture thereof which is bulky, less deforming and gives less chemical stimulations. Specifically, they include an absorbent body having a basis weight of from 100 to 2000 g/m² and a volume of from 1 to 50 mm formed by mixing from 80 to 100% of pulp and from 20 to 0% of a polymeric absorbent body, covering the same with tissue paper and sheeting the same by embossing. For the manufacturing method of the absorbent body, those sheeted by an air-laid method, spun lacing method, paper making method, melt blowing method, etc. may be applied with needling or embossing by passage between rolls such as of a dot-shape, lattice-shape or corrugated shape. Further, also for preventing the deformation of the absorbent body, the emboss area ratio is within a range from 10 to 100%, preferably, within a range from 30 to 80%.

In order that the combinational sheet conforms easily with a crotch shape, an emboss pattern tracing the crotch shape, so-called a hinge may be formed to the top sheet and the absorbent body. Further, in a case where a sheet applied with a releasing treatment is disposed between the combinational sheet and the individual packaging body, the sheet comprising paper prepared by a paper making method is preferred.

<External Container>

The materials for the external container for incorporating the first individual packaging body that incorporates the inter-labial pad and the second individual packaging body that incorporates the combinational sheet together include films constituted with polyethylene, polypropylene, polyethylene terephthalate, polylacitc acid and polybutyl succinate, non-woven fabrics or paper as well as those materials applying lamination thereon, or those materials vapor-deposited with aluminum, plastics, etc.

The invention can be utilized in a packaging body incorporating both the inter-labia pad and the combinational sheet used together with the inter-labial labial pad in which the state of using the inter-labia pad and the combinational sheet together can be recognized without opening the seal.

1. A wrapping body incorporating an inter-labial pad as a sanitary article and a combinational sheet used together with the inter-labial pad by wrapping them integrally into an external container, wherein a first individual wrapping body comprising the inter-labial pad incorporated in an individual wrapping container for the inter-labial pad and a second individual wrapping body comprising the combinational sheet incorporated in an individual wrapping container for the combinational sheet are combined each by one or more and incorporated in the external container, and wherein an indication means indicating the state of combined use of the inter-labial pad and the combinational sheet are disposed to the external container at a position that can be recognized by a wearer.

2. The wrapping body according to claim 1, wherein the indication means related with the incorporated sanitary article is provided to the individual wrapping container for the inter-labial pad and/or the individual wrapping container for the combinational sheet.

3. The wrapping body according to claim 1, wherein the wrapping body further comprises a first intermediate wrapping container incorporating only the first individual wrapping bodies in plurality and a second intermediate wrapping container incorporating only the second individual wrapping bodies in plurality, and the indication means showing the method of use related to the incorporated sanitary article is provided to the first intermediate wrapping container and/or the second intermediate wrapping container.

4. A wrapping body in which a first individual wrapping body incorporating an inter-labial pad and a second individual wrapping body incorporating a combinational sheet used together with the inter-labial pad are incorporated in combination each by one or more in an external container, wherein a transparent region is formed to the external container through which at least a portion of the incorporated first individual wrapping body and second individual wrapping body can visually recognize.

5. The wrapping body according to claim 4, wherein the transparent region has a design schematically expressing the convex and concave profile of a body portion to which the inter-labial pad is put.

6. The wrapping body according to claim 5, wherein the design is constituted by providing a not transparent or semi-transparent portion in the transparent region.

7. The wrapping body according to claim 5, wherein the first individual wrapping body and the second individual wrapping body are each in a substantially plate shape, the convex and concave of the body portion expresses labia, the first individual wrapping body is arranged so as to be up-righted relative to a convex portion corresponding to a pudendal slit, and the second individual wrapping body is arranged so as to be turned sideway relative to the convex portion.

8. A wrapping body in which a first individual wrapping body for incorporating an inter-labial pad to be inserted between labia and a second individual wrapping body for incorporating a combinational sheet to be used together with the inter-labial pad are incorporated in combination each by one or more in an external container, wherein the positions for incorporating the first individual wrapping body and the second individual wrapping body are determined in relation with the order of using the inter-labial pad and the combinational sheet.

9. The wrapping body according to claim 8, wherein the combinational sheet is put on prior to the inter-labial pad, the external container has a seal-opening means for opening the seal of the external container and the second individual wrapping body is incorporated being disposed at a position nearer to the seal-opening means than the first individual wrapping body.
